(19) United States
(12) Patent Application Publication
(10) Pub. No.: US 2011/0014295 A1
(43) Pub. Date: Jan. 20, 2011

(54) METHOD OF DETERMINING THE WEIGHT OF THE COATING TO BE APPLIED TO FORM A CONTROLLED RELEASE DOSAGE FORM

(75) Inventors: Orapin Rubino, Towaco, NJ (US); David Jones, Ramsey, NJ (US); Robert A. Femia, Kinnelon, NJ (US); Oliver Mueller, Kinnelon, NJ (US); Narayan Rangunathan, West Nyack, NY (US); Norbert Pollinger, Mullheim (DE); Armin Prasch, Freiburg (DE); Anne Ettner, Wollbach-Kandern (DE)

Correspondence Address: HEDMAN & COSTIGAN, P.C., 1230 AVENUE OF THE AMERICAS, 7th floor, NEW YORK, NY, 10020, US

(73) Assignee: Glatt Air Techniques, Inc., Ramsey, NJ (US)

(21) Appl. No.: 12/449,667

(22) PCT Filed: Feb. 6, 2008
(86) PCT No.: PCT/US2008/001639

§ 371 Date: Sep. 2, 2010

Related U.S. Application Data
(60) Provisional application No. 60/903,224, filed on Feb. 23, 2007.

(51) Int. Cl. A61K 9/14; A61K 31/138

(52) U.S. Cl. 424/490; 514/652

(57) ABSTRACT

A method of coating spherical particles of a beta blocker compound which is based on
(a) determining the surface area of a weighed sample of said spherical particles;
(b) determining the weight of coating material per surface area unit of spherical particles that will provide a desired release profile for said beta-blocker;
(c) determining the surface area of a subsequent batch of spherical particles containing the same beta;
(d) coating the subsequent batch of spherical particles with the coating applied in step (b) to provide a coating having substantially the same weight of coating material per surface area unit of spherical particles as determined in step (b).

### BACKGROUND OF THE INVENTION

The formulation of controlled release membranes by the use of liquid coating systems is well known. In the prior art it is known that it is difficult to consistently manufacture batches of membrane coated products that have the same release profiles both in vitro and in vivo. This is a particular problem with drugs such as beta blockers that have a narrow therapeutic index which makes it essential that the vivo and in vitro release profiles are consistent from batch to batch. For this reason, the art has developed sophisticated apparatus and process controls for the rate, temperature, humidity and times that are employed for forming semi-permeable membranes on solid particles that contain active chemicals such as pharmaceuticals, seeds, fertilizers and the like. Even with careful process controls, it is sometimes necessary to reprocess or discard multiparticulate controlled release products due to a lack of conformance with established release rate profiles.

The amount of a coating agent to provide a desired therapeutic effect may be determined using conventional experimental techniques that measure the release rate of a pharmaceutical from a particular membrane system by trial and error using in vitro and in vivo procedures. These methods are based on the use of preset process controls that do not take into account any variations in the size or size distribution curve for the multiparticulate particles that are to be coated.

In Aqueous Polymeric Coatings for Pharmaceutical Dosage Forms, J. W. McGinity Ed., Marcel Dekker. Inc., NY (1989), pp. 350-355, which is incorporated by reference, it was disclosed that surface rugosity might have a possible effect on the release rate of a drug contained in membrane coated particles. The concept that was disclosed was that particles of equivalent sphere diameters would have different surface areas depending on the smoothness of the surface of the spheres. No information was given as to how to reproducibly prepare batches of coated spherical particles that will release active ingredients at reproducible rates. In Pharmaceutical Pelletization Technology, Isaac Ghebre-Sellassie Ed., Marcel Dekker, Inc., NY (1989), pp. 246-252, which is incorporated by reference, it was disclosed that rough pellets have a greater surface area than smooth pellets and when the same weight of a coating is applied to separate batch of smooth pellets and a separate batch of rough pellets, the smooth pellets will exhibit a faster rate of release than the rough pellets.

U.S. Pat. No. 5,527,545 describes a liquid suspension of enteric coated pellets of naproxen, which is a drug having a low solubility in water. In the course of making the multiple coated naproxen pellets, the surface area of a batch of pellets is used as a basis apply a constant amount of coating so that the coating is directly proportional to the surface area. No drug other than naproxen is mentioned by U.S. Pat. No. 5,527,545 and the naproxen pellets range in size from 50 to 500 μm and each pellet is provided with four separate coatings at least one of which is an enteric coating. U.S. Pat. No. 4,138,475 discloses a specific propranolol controlled release formulation.

The present invention provides a reproducible method for making controlled release pellets containing a beta-blocker compound where a single membrane is applied to a pelleted formulation, where the weight of the coating is based on the surface area of the pellet and the weight of the coating is determined by selecting a coating weight that is proportional to the surface area of the beta-blocker pellets, based on a weight per surface area of the beta-blocker pellets, previously determined in vivo or in vitro release profile. The present invention is particularly applicable to beta-blockers such as propranolol which have a high solubility in water.

### SUMMARY OF THE INVENTION

The invention provides a method of coating substantially spherical particles comprising a beta-blocker compound where the pellets are substantially free of surface defects, with a membrane forming coating to provide a membrane on said substantially spherical particles that controls the release of an active compound from said substantially spherical particle at a predetermined level. The method comprises the following steps:

(a) determining the surface area of a weighed sample of said substantially spherical particles in a first batch of substantially spherical particles based on the particle size distribution and density;
(b) determining the weight of coating material per surface area unit of said substantially spherical particles that will provide a coating level of a composition for said substantially spherical particles of step (a) that will provide a desired release profile in vivo;
(c) determining the surface area of a new batch of substantially spherical particles containing the same active compound that was contained in the substantially spherical particles that were coated in step (b);
(d) coating said new batch of substantially spherical particles with the coating applied in step (b) to provide a coating on said new batch of substantially spherical particles, wherein said coating on said new batch of spherical particles has the substantially the same weight of coating material per surface area unit of substantially spherical particles as determined in step (b).

Accordingly, it is a primary object of the invention to provide a method of producing controlled release substantially spherical particles of a beta-blocker which have a consistent in vitro and in vivo release profiles.

It is also an object of the invention to provide a method manufacturing controlled release multiparticulate particles of a beta-blocker which avoids the necessity of reworking or discarding the coated particles because they fail to meet established release rate profiles.

These and other objects of the invention will become apparent from their appended specification.

The term beta-blocker is used to describe those pharmaceutical compounds that are classified as beta-adrenergic receptor blocking agents such as propranolol, metoprolol, +timolol, oxpremolol, alpremolol, pindolol, sotalol, alebutolol, atenolol, and the like. The release rates are expressed in terms of weight percent of the total active compound in the dosage form which is released after a measured lapse of time in accordance with standard procedures that are well know in the art.

### DETAILED DESCRIPTION OF THE INVENTION

In the practice of the present invention, it is contemplated that a multiparticulate controlled release formulation of a beta-blocker will have been made and tested for either a desired pharmacological effect or for the purpose of providing a formulation of the same beta-blocker that is bioequivalent to a previously approved controlled release formulation. Once a particular beta-blocker has been approved for full scale manufacturing, the present invention may be utilized to provide a coating parameter that will result in a method of manufacturing a multiparticulate formulation of the beta-blocker that has a consistent release profile.

The ratio of the weight of coating per unit surface area for a particular beta-blocker and a particular membrane based release controlling system will be unique for that product with regard to the particular release rate profile for that product whether it is in vivo or in vitro. The invention is particularly useful with thin coatings of controlled release membranes.

Once an operable system for making a multiparticulate controlled release formulation has been identified, the present invention may be utilized to provide a manufacturing method that will provide sufficient batch to batch uniformity that it will avoid the need to destroy particular batches which are out of specification due to failed release rate tests.

Thus, when a release controlling system is chosen for a particular beta-blocker, it may be experimentally determined which type of a membrane forming system and adjuvants will provide the desired controlled release system for the particular beta-blocker. The coating ratio that is determined for that particular system will be unique for that system and it will be necessary to determine a coating ratio for each beta-blocker release system. The invention is particularly useful when substantially spherical particles are made that are substantially free of surface defects. These spherical particles may be made using techniques such as seed coating using sugar seeds or other solid bead forming materials, (i.e microcrystalline cellulose pellets such as Cellets) spheronization, microtabletting techniques, or by the use of the CPS techniques disclosed in U.S. Pat. No. 6,449,869; U.S. Pat. No. 6,354,728; and U.S. 2004/0185111, all of which are incorporated by reference. The term “substantially spherical” is used to describe spherical particles useful in the present invention. These particles will have an aspect ratio, which is the ratio of the shortest axis of the particle to the longest axis of the particle, of from 0.2-1. It is to be understood that a perfect sphere will have an aspect ratio of 1.

After an operable formulation has been made using conventional coating techniques based on the use of membrane forming polymers that are applied to the spherical particles using conventional layering techniques and it has been decided to produce large scale quantities of such a formulation, the first step is to determine the surface area of the substantially spherical particles which comprise the beta-blocker, after they have been coated or layered with the beta-blocker. The specific surface area may be determined by methods such as measurement of the pellet coupled with a determination of the average particle size distribution, or by gas adsorption or air permeability. Pharmaceutical Pelletization Technology, Isaac Ghebre-Sellassie Ed., Marcel Dekker, Inc., NY (1989), pp. 246-252, discloses various techniques for determining the surface area of pellets.

The surface area is measured to provide a baseline of total surface area of the uncoated substantially spherical microparticles containing a beta-blocker, which are to be coated to give a particular release profile, when the multiparticulate composition is subsequently coated and tested to establish a release profile. The next step comprises determining the quantity of coating material, that is necessary to provide a controlled release multiparticulate beta-blocker formulation having the desired release profile, and then using the surface area and total weight (solids) that will provide the desired release profile to calculate the ratio of surface area to the weight (solids) of controlled release forming composition. The weight of the controlled release coating per unit of surface area is used to determine how much controlled release formulation is to be applied to the multiparticulate formulation of the beta-blocker.

A formula that may be used to calculate the specific surface area is

$$Specific Surface area = \frac{6}{true density \times mean volume surface diameter of the spheres} .$$

The preferred coating weight is between about 0.05 to 3 mg/cm² and the especially preferred coating weight is between 0.1 to 1 mg/cm² of surface area of multiparticulate particles. The coating conditions will vary depending on the materials that are employed and these conditions may be determined using conventional techniques.

The total applied coating weight is preferably from 1 to 30 g/100 g of starting material. and especially preferably from 1 to 10 g/100 g of starting material.

The particle sizes should be between 100 and 1400 microns in diameter, preferably from 600 to 1200 microns in diameter; and especially preferably from 700 to 1100 microns in diameter.

The particular controlled release coatings may be selected from those which are commerically available as FDA approved materials for use in pharmaceuticals when formulating a pharmaceutical or other compositions. Examples of useful materials are described in Aqueous Polymeric Coatings for Pharmaceutical Dosage Forms, J. W. McGinity Ed., Marcel Dekker. Inc., NY (1989), pp 1-153, which is incorporated by reference. Generally, in the process of the invention, it has been found that unplasticized coatings that are not enteric coatings are preferred. Ethyl cellulose is a preferred coating material for use in the invention

### Example I

A 70 kg. batch of propranolol HC1 core pellets (60% propranolol-40% microcrystalline cellulose) were produced using a direct pelletization process (U.S. Pat. No. 6,449,869; U.S. Pat. No. 6,354,728; and U.S. 2004/0185111). The propranolol and the microcrystalline cellulose were prewetted and loaded into an apparatus as described in U.S. Pat. No. 6,449,869 and U.S. Pat. No. 6,354,728. Water is applied until pellets of the desired size of approximately 600 to 1000 micron were formed. The pellets were dried in a fluid bed drier at a inlet air temperature of 60° C. The pellets were coated in the GPCG-30 (Glatt Fluid Bed Processor, equipped with a 18″ Wurster HS SRS (bottom spray) insert. The target coating level for propranolol core pellets was determined to be 0.41 mg/cm² to obtain desirable release profile The coating polymers were ethyl cellulose and hypromellose in organic solvents.

Data from coating of five different Propranolol HC1 core pellet batches were summarized in Table 1. Using specific surface area coating concept to adjust quantity of coating material for each batch of core pellets, results in reproducible dissolution profiles of the coated pellets. In all five coating batches, yield was greater than 97%.

TABLE 1

| 1. Starting Materials | | | | | |
|---|---|---|---|---|---|
| 1.1 Propranolol HCl Core Pellets | | | | | |
| Batch # Propranolol Core Pellets GAT | A | B | C | D | E |
| Real density [g/cm3] | 1.322 | 1.3362 | 1.3379 | 1.3359 | 1.3348 |
| Sauter Diameter | 765.53 | 778.79 | 771.29 | 747.39 | 782.08 |
| Specific Surface Area [cm²/g] | 59.3 | 57.7 | 58.1 | 58 | 57.5 |
| Theoretical Coating per kg Core pellets | 486.26 | 473.14 | 476.42 | 475.6 | 471.5 |
| [g/kg] | | | | | |
| Amount of liquid to be processed [kg] | 33.72 | 33.14 | 33.4 | 33.32 | 33.03 |
| 2. Process parameters | | | | | |
| Spray Time | n.a. | 157 | 160 | 156 | 167 |
| Drying Time | 5 + 5 | 10 | 10 | 10 | 10 |
| Spray pressure | 2.5 | 2.5 | 2.5 | 2.5 | 2.5 |
| Air volume [cm3/h] | 736-767 | 750 | 746-767 | 744-780 | 745-775 |
| Product Temperature during | 37.6-39.2 | 38.5-39.2 | 38.3-39.7 | 38.6-40.0 | 38.2-39.4 |
| spraying(Min/Max) | | | | | |
| 3. Coated Propranolol HCl Pellets | | | | | |
| Yield [%] | 99.17 | 97.26 | 97.55 | 97.43 | 97.5 |
| Coated Batch Numbers | AC | BC | CC | DC | EC |
| Dissolution Results | | | | | |
| Dissolution Propranolol HCl after 90 | 23.6 | 23.6 | 24.9 | 26.2 | 28.2 |
| min [%] | | | | | |
| Dissolution Propranolol HCL after 240 | 53.1 | 51.7 | 55.7 | 56.8 | 58.8 |
| min [%] | | | | | |
| Dissolution Propranolol HCL after 480 | 73.8 | 72.5 | 77.8 | 78.3 | 79.7 |
| min [%] | | | | | |

The Sauter diameter is determined by a laser light scattering method using a Malvern Particle Analyzer.

A preferred propranolol hydrochloride formulation will have the following release profile when tested in Apparatus 1, according to U.S.P. 30, Method B for a delayed release dosage form using 900 ml of pH 1.2 buffer solution for 1.5 hours during the acid stage using acceptance criteria given under Acceptance Table 3. For the buffer stage, 900 ml of pH6.8 buffer solution is used for the remainder of the test. Samples are analyzed using UV absorption at 320 nm with respect to a baseline drawn from 355 nm through 340 nm with a propranolol reference standard.

| Hours | Amount dissolved |
|---|---|
| 1.5 | 15-30% |
| 4 | 40-60% |
| 8 | 65-80% |

1. A method of coating substantially spherical particles which comprise a beta blocker compound, said substantially spherical particles being substantially free of surface defects and having a membrane forming coating to provide a membrane on said substantially spherical particles that controls the release of said beta-blocker from said substantially spherical particles at a predetermined release rate, said method comprising:
(a) determining the surface area of a weighed sample of said substantially spherical particles in a batch of substantially spherical particles;
(b) determining the weight of coating material per surface area unit of substantially spherical particles that will provide a coating level of a composition for said substantially spherical particles of step (a) that will provide a desired release profile for said beta-blocker;
(c) determining the surface area of a subsequent batch of substantially spherical particles containing the same beta blocker that was contained in the substantially spherical particles that were coated in step (b);
(d) coating said subsequent batch of substantially spherical particles with the coating applied in step (b) to provide a coating on said subsequent batch of substantially spherical particles, wherein said coating on said new batch of spherical particles has the substantially the same weight of coating material per surface area unit of substantially spherical particles as determined in step (b).

2. A method as defined in claim 1 wherein the in vitro release profile for the substantially spherical particles coated in step (b) is determined.

3. A method as defined in claim 1 wherein the in vivo release profile for the substantially spherical particles coated in step (b) is determined.

4. A method as defined in claim 1 where the active compound is a beta blocker.

5. A method as defined in claim 1 where the beta blocker is selected from the group consisting of propranolol, metoprolol, timolol, oxpremolol, alpremolol, pindolol, sotalol, alebutolol and atenolol and the like

6. A method as defined in claim 5 where the beta blocker is selected from the group consisting of propranolol, metoprolol, timolol, oxpremolol, alpremolol, pindolol, sotalol, alebutolol and atenolol and the like

7. A method as defined in claim 1 where the surface area is the nominal surface area and the applied total coating weight is between 0.05 to 3 mg/cm².

8. A method as defined in claim 7 where the surface area is the nominal surface area and the applied total coating weight is between 0.1 to 1 mg/cm².

9. A method as defined in claim 1 where the total applied coating weight is from 1 to 30 g/100 g of starting material.

10. A method as defined in claim 9 where the total applied coating weight is from 1 to 10 g/100 g of starting material.

11. A method as defined in claim 1 where the particle sizes are between 100 and 1400 microns in diameter.

12. A method as defined in claim 11 where the particle sizes are between 600 and 1200 microns in diameter.

13. A method as defined in claim 12 where the particle sizes are between 700 and 1100 microns in diameter.

14. A method of coating substantially spherical particles comprising propranolol hydrochloride that are substantially free of surface defects with a membrane forming coating to provide a membrane on said substantially spherical particles that controls the release of propranolol hydrochloride from said substantially spherical particles at a predetermined release rate, said method comprising:
(a) determining the surface area of a weighed sample of said substantially spherical particles in a batch of substantially spherical particles based on the particle size distribution;
(b) determining the weight of coating material per surface area unit of substantially spherical particles that will provide a coating level of a composition for said substantially spherical particles of propranolol hydrochloride in step (a) that will provide a desired release profile for said propranolol hydrochloride in vitro;
(c) determining the surface area of a subsequent batch of substantially spherical particles containing propranolol hydrochloride that was contained in the substantially spherical particles that were coated in step (b);
(d) coating said subsequent batch of substantially spherical particles with the coating applied in step (b) to provide a thin coating on said subsequent batch of substantially spherical particles, wherein said thin coating on said subsequent batch of spherical particles has the substantially the same weight of coating material per surface area unit of substantially spherical particles as determined in step (b).
